(19) United States
(12) Patent Application Publication
(10) Pub. No.: US 2025/0194920 A1
(43) Pub. Date: Jun. 19, 2025

(54) VISTA DE-NOISING

(71) Applicant: TOPCON CORPORATION, Tokyo (JP)

(72) Inventors: Zaixing MAO, Tokyo (JP); Zhenguo WANG, Ridgewood, NJ (US); Kinpui CHAN, Ridgewood, NJ (US)

(21) Appl. No.: 18/846,509

(22) PCT Filed: Mar. 13, 2023
(86) PCT No.: PCT/US2023/064227

§ 371 Date: Sep. 12, 2024

Related U.S. Application Data
(60) Provisional application No. 63/269,307, filed on Mar. 14, 2022.

(51) Int. Cl. A61B 3/12; A61B 3/10; A61B 3/14; G06T 5/50; G06T 5/70; G06T 7/00; G16H 30/40

(52) U.S. Cl. CPC A61B 3/1233; A61B 3/102; A61B 3/14; G06T 5/50; G06T 5/70; G06T 7/0012; G16H 30/40; G06T 2207/10101; G06T 2207/20221; G06T 2207/30041; G06T 2207/30104

(57) ABSTRACT

An optical coherence tomography angiography (OCT-A) method that includes generating at least two OCT-A images based on different interscan times, de-noising the at least two OCT-A images, and generating a short interscan time (SIT) representative image and a long interscan time (LIT) representative image based on the at least two de-noised OCT-A images. Estimating a relative blood flow velocity based on the SIT representative image and the LIT representative image. Further, generating a blood flow image based on the relative blood flow velocity.

### CROSS-REFERENCE TO RELATED APPLICATIONS

This application claims priority to U.S. Provisional Patent Application No. 63/269,307 filed Mar. 14, 2022 and entitled “VISTA DE-NOISING”, the entirety of which is incorporated herein by reference.

### BACKGROUND

Optical coherence tomography (OCT) is a non-invasive imaging technique, often used in ophthalmology. OCT relies on principles of interferometry to image and collect information about an object (such as the eye of a subject). Particularly, light from a source is split into a sample arm where it is reflected by the object being imaged, and reference arm where it is reflected by a reference object such as a mirror. The reflected lights are then combined in a detection arm in a manner that produces an interference pattern that is detected by spectrometer, photodiode(s) or the like. The detected interference signal is processed to reconstruct the object and generate OCT images.

More particularly, structural OCT images and volumes are generated by combining numerous depth profiles (A-lines, e.g. along a Z-depth direction at an X-Y location) into a single cross-sectional image (B-scan, e.g., as an X-Z or Y-Z plane), and combining numerous B-scans into a volume. These depth profiles are generated by scanning along the X and Y directions. En-Face images in the X-Y plane may be generated by flattening a volume in all or a portion of the Z-depth direction, and C-scan images may be generated by extracting slices of a volume at a given depth. Angiographic (OCT-A) images may be generated by comparing information from structural images and/or volumes at different times (e.g., from repeated scans). Assuming the structure of the object remains the same in a relatively short time period between scans (on the order of milliseconds to seconds), the changes may be attributed to blood flow from which vasculature may be identified.

As is with many imaging techniques, unwanted signal noise can be common in OCT and OCT-A imaging. For example, OCT and OCT-A images may be prone to noise and artifacts caused by variations in flow speed, signal quality, and patient movements. The presence of such noise makes is difficult to distinguish blood vessel structure and blood flow speeds, particularly among small capillaries. While some techniques exist for removing/mitigating noise, these techniques often require a larger number of scans, and thus increase total scan time and processing requirements.

### BRIEF SUMMARY

According to one example of the present disclosure, a method comprises: generating at least three structural optical coherence tomography (OCT) images of a same location of an object; generating at least two OCT-Angiography (OCT-A) images based on the structural OCT images, the at least two OCT-A images being based on different interscan times between the corresponding structural OCT images from which the OCT-A images were generated; de-noising the at least two OCT-A images; generating a short interscan time (SIT) representative image and a long interscan time (LIT) representative image based on the at least two OCT-A images; estimating a relative blood flow velocity based on the SIT-representative image and the LIT-representative image.

In various embodiments of the above example, the at least two OCT-A images are cross-sectional B-scans; the method further comprises: generating the at least two OCT-A images for a plurality of locations of the object, thereby forming a plurality of OCT-A volumes, and subsequent to de-nosing the at least two OCT-A images, de-noising an en-face image of each of the plurality of OCT-A volumes, wherein the SIT-representative image and the LIT-representative image are based on the denoised en-face images; the method further comprises generating a blood flow image based on the estimated relative blood flow velocity; the blood flow image is a color-mapped image in which pixel color corresponds to the estimated a relative blood flow velocity; the de-noising is performed by at least one trained machine learning system; generating the SIT-representative image comprises statistically combining de-noised OCT-A images having an interscan time less than a predetermined threshold, and generating the LIT-representative image comprises statistically combining de-noised OCT-A images having an interscan time greater than the predetermined threshold; the estimated relative blood flow velocity at a given location is a ratio of the SIT-representative image at the given location to the LIT-representative image at the given location; estimating the relative blood flow velocity is a pixel-wise determination of the ratio of the SIT-representative image to the LIT-representative image; the ratio is raised to a power greater than or equal to 1.5; and/or the object is a retina.

According to another example of the present disclosure, a method comprises: generating a plurality of optical coherence tomography angiography (OCT-A) volumes, each of the plurality of OCT-A volumes being based on different interscan times between structural OCT images from which the OCT-A volumes were generated; de-noising the plurality of OCT-A volumes by: de-noising B-scan images from the plurality of OCT-A volumes; and subsequent to de-noising the B-scan images, de-noising en-face images from the plurality of OCT-A volumes; generating a short interscan time (SIT) representative image by statistically combining de-noised en-face images from OCT-A volumes having an interscan time less than a predetermined threshold; and generating a long interscan time (LIT) representative image by statistically combining de-noised en-face images from OCT-A volumes having an interscan time greater than the predetermined threshold.

In various embodiments of the above example, the method further comprises: estimating a relative blood flow velocity based on the SIT-representative image and the LIT-representative image, and generating a blood flow image based on the estimated relative blood flow velocity; the de-noising is performed by at least one trained machine learning system; the method further comprises: estimating a relative blood flow velocity as a pixel-wise determination of a ratio of the SIT-representative image at the given location to the LIT-representative image raised to a power greater than or equal to 1.5, and generating a blood flow image based on the estimated relative blood flow velocity; and/or the object is a retina.

### BRIEF DESCRIPTION OF THE SEVERAL VIEWS OF THE DRAWING

FIG. 1 illustrates an example schematic of an optical coherence tomography system of the present disclosure.

FIG. 2 illustrates an example method of the present disclosure.

FIG. 3 illustrates an example noise reduction method of the present disclosure.

FIG. 4 illustrates an example noise reduction method of the present disclosure.

FIG. 5 illustrates an example image depicting blood flow velocity.

### DETAILED DESCRIPTION OF THE DRAWING

Considering the above, the present disclosure relates to OCT-A noise reduction techniques while reducing the total scan time. More particularly, the present disclosure relates to utilizing machine learning systems for OCT-A image de-noising and estimating relative blood flow speed.

Using the methods described below, OCT-A images can have better noise suppression without the use of filters or the like. With better noise suppression, such OCT-A images can illustrate vasculature in better detail and be used to identify specific locations of slow and fast blood flow. Generating color-mapped images utilizing the described ratio can produce better dynamic range and an estimated relative blood flow speed can be determined.

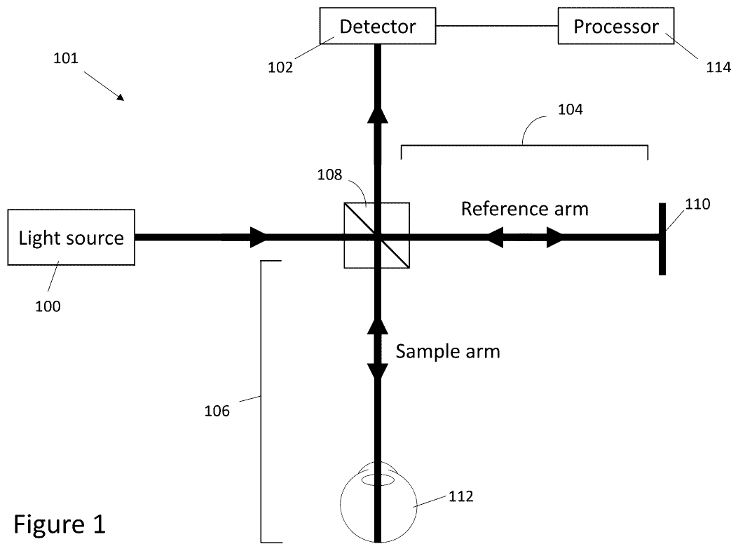

The present disclosure can utilize an OCT system 101, such as that illustrated in FIG. 1. As discussed above the system 101 includes a light source 100. The light generated by the light source 100 is split by, for example, a beam splitter (as part of interferometer optics 108), and sent to a reference arm 104 and a sample arm 106. The light in the sample arm 106 is backscattered or otherwise reflected off an object, such as the retina of an eye 112. The light in the reference arm 104 is backscattered or otherwise reflected, by a mirror 110 or like object. Light from the sample arm 106 and the reference arm 104 is recombined at the optics 108 and a corresponding interference signal is detected by a detector 102. The detector 102 can be a spectrometer, photo detector, or any other light detecting device. The detector 102 outputs an electrical signal corresponding to the interference signal to a processor 114, where it may be stored and processed into OCT signal data and/or OCT-A data. The processor 114 may then further generate corresponding images or otherwise perform analysis of the data. The processor 114 may also be associated with an input/output interface (not shown) including a display for outputting processed images, or information related to the analysis of those images. The input/output interface may also include hardware such as buttons, keys, or other controls for receiving user inputs to the system. In some embodiments, the processor 114 may also be used to control the light source and imaging process.

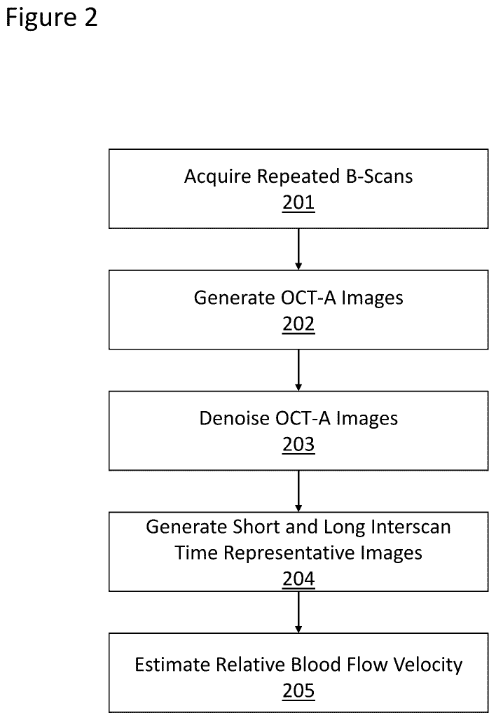

With reference to FIG. 2, an example method of the present disclosure first acquires two or more repeated B-scans at the same location of an object 201. These B-scans can be acquired, for example, using the OCT system 101 illustrated in FIG. 1 by capturing a plurality of A-lines and generating structural OCT images and volumes therefrom with the processor 114. Particularly, these structural OCT images (e.g., B-scans) are generated by combining a plurality of depth profiles (A-lines) into a cross sectional image (B-scan), and the volumes result from a plurality of combined B-scans. Similarly, the processor 114 can generate en-face images by flattening a volume in the depth direction or C-scans by extracting slices of a volume at a given depth.

The repeated B-scans may be obtained by any scanning protocol. For example, an entire OCT volume may be acquired before acquiring repeated data from any location within the volume. In other embodiments, individual A-lines or B-scans may be repeated prior to advancing to the next A-line or B-scan. In this manner, multiple B-scans and/or volumes are effectively acquired simultaneously.

The processor 114 uses the at least two repeated B-scans per location to generate an OCT-A image 202 of that location. While the method is possible with two repeated B-scans, the OCT system 101 could generate more B-scans. OCT-A images are generated for each pair of images at a given location, regardless of the number of B-scans. These comparisons may be based on any or all possible combinations of B-scans. According to one example, variable interscan time analysis (VISTA) methods can be used to generate OCT-A images. By way of example, these OCT-A images may be generated as described in U.S. Pat. No. 10,839,515, titled SYSTEMS AND METHODS FOR GENERATING AND DISPLAYING OCT ANGIOGRAPHY DATA USING VARIABLE INTERSCAN TIME ANALYSIS, the entirety of which is incorporated herein by reference. As above with the structural OCT images, combining a plurality of OCT-A images from a plurality of cross-sectional locations can form an OCT-A volume.

More particularly, VISTA involves generating OCT-A images corresponding to different interscan times, and then interpreting the differences in these images/data as being related to blood flow speed, velocity, or related quantities. The speed of the OCT-A system also may also affect the acquisition of the blood flow, for example, if the OCT-A system has a fast A-scan rate (e.g., 400 kHz), it may be difficult to capture slow blood flow.

For example, given four repeated B-scans B1-B4 at times t1-t4, OCT-A images may be generated for the pairs B1-B2, B1-B3, B1-B4, B2-B3, B2-B4, and B3-B4. Thus, four repeated B-scans may result in the generation of six OCT-A images. The time between repeated OCT B-scans is herein referred to as the “interscan time.” In the above example, assuming a constant time Δt between OCT B-scans, the OCT-A images may have interscan times of Δt (e.g., t2-t1) for OCT-A images based on OCT B-scan pairs B1-B2, B2-B3, and B3-B4, of 2Δt (e.g., t3-t1) for OCT-A images based on OCT B-scan pairs B1-B3 and B2-B4, and of 3Δt (e.g., t4-t1) for the OCT-A image based on OCT B-scan pair B1-B4.

The interscan time determines the sensitivity and saturation of the OCT-A signal (and image) versus blood flow speed. In other words, longer interscan times are more sensitive to slow flow speeds, but result in saturated OCT-A signals if flow is fast. Shorter interscan times can differentiate between these faster flows, but generally show reduced OCT-A signals and may not detect blood flows having slower speeds. The relationship between the OCT-A signal and blood flow velocity is approximately linear. For example, doubling the interscan time will result in, approximately, the same change in an OCT-A signal as doubling the blood flow velocity. This relationship can be exploited to estimate blood flow velocity and/or related quantities.

Following OCT-A image generation 202, the processor 114 can de-noise the OCT-A images 203. De-noising can be achieved by various machine learning techniques, for example, spatial filtering, temporal accumulation, deep learning reconstruction, or the like. The de-noising process can comprise one or more levels of de-noising. In some embodiments, noise reduction is accomplished by applying a deep-learning based noised reduction technique, such as that described in U.S. Pat. No. 11,257,190, titled IMAGE QUALITY IMPROVEMENT METHODS FOR OPTICAL COHERENCE TOMOGRAPHY, the entirety of which is incorporated herein by reference. Further, shadow and projection artifacts may be reduced by applying image-processing and/or deep-learning techniques, such as that described in U.S. Pat. No. 11,361,481, titled 3D SHADOW REDUCTION SIGNAL PROCESSING METHOD FOR OPTICAL COHERENCE TOMOGRAPHY (OCT) IMAGES, the entirety of which is incorporated herein by reference.

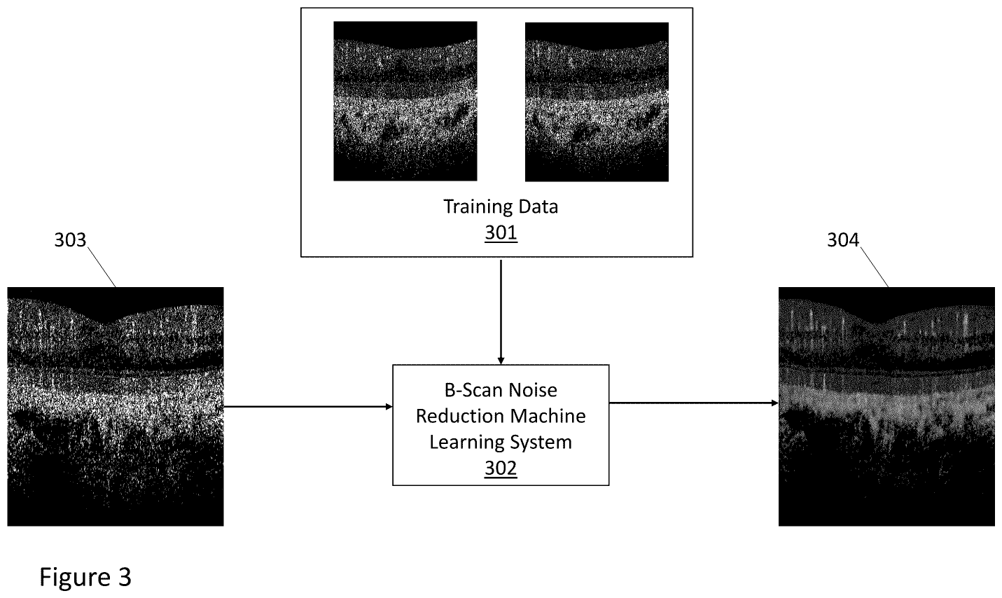

For example, as illustrated in FIG. 3, an OCT-A volume can be de-noised at a B-scan level. In other words, OCT-A cross-sectional B-scans 303 are input to a B-scan noise reduction machine learning system 302, which is trained to output a de-noised B-scan 304. The B-scan noise reduction machine learning system 302 can be trained using various machine learning training techniques, for example, supervised, semi-supervised, unsupervised, reinforcement, or the like. Training data 301 of the machine learning system can include pairs of B-scan OCT-A images taken at the same location, for example, where one image contains noise and the other is de-noised.

In other embodiments, paired cross-sectional OCT-A B-scans from a common location, neither being de-noised, are input as training data 301. In this way, the machine learning system learns to recognize random noise between the pair of OCT-A training images. This recognized random noise can then be removed from other input OCT-A B-scans to output de-noised OCT-A B-scans. In other words, the machine learning system 302 can be trained to recognize noise in an OCT-A image by providing the machine learning system 302 training data 301 comprising pairs of OCT-A images representing the same location. Because any structural differences between the OCT-A images are already accounted for by the OCT-A process, the differences between the OCT-A images can simply be considered noise.

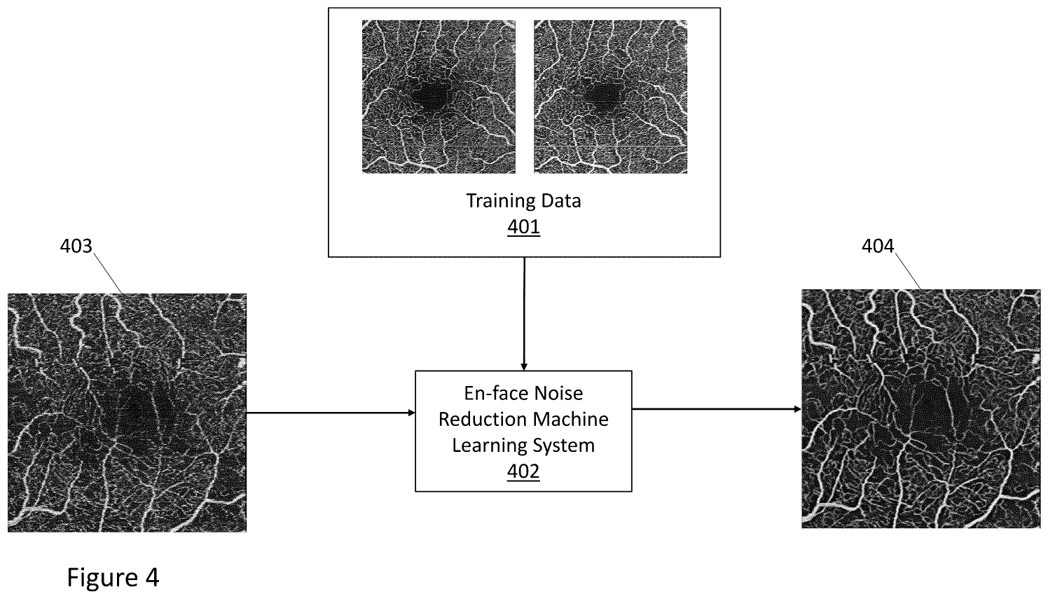

Similarly, as illustrated in FIG. 4, noise reduction can occur at an en-face level. The en-face noise reduction can be accomplished in a similar manner to that discussed above with respect to B-scans. Depending on the embodiment, en-face images from multiple depths (or depth ranges) can be de-noised for a single OCT-A volume. For example, an en-face image 403 from an OCT-A volume can be input into an en-face noise reduction machine learning system 402, which outputs a de-noised en-face image 404. The machine learning system 402 can be trained to recognize and remove noise from an inputted en-face image 403. The en-face AI noise reduction system can be trained using various machine learning training techniques, for example, supervised, semi-supervised, unsupervised, reinforcement, or the like. As with the B-scan noise reduction system, training data 401 can include pairs of en-face images taken at the same location, where one image contains noise and the other image is de-noised. In other embodiments, training data 401 may include pairs of en-face images having random noise, where the difference between each image is the random noise.

The noise reduction process can be implemented in various ways. For instance, in one embodiment, the noise reduction process 203 can first de-noise B-scans of an OCT-A volume (e.g., with B-scan noise reduction machine learning system 302), and then perform en-face level noise reduction (e.g., with en-face noise reduction machine learning system 402). In other words, de-noised B-scans can be combined into an en-face image for en-face level de-noising. In other embodiments, the noise reduction process 203 first de-noises an OCT-A volume at the en-face level prior to de-noising at the B-scan level. In still other embodiments, an OCT-A volume may be separately de-noised at the B-scan level and at the en-face level. In these cases, the resulting B-scan level de-noised volume and en-face level de-noised volume can be recombined in any statistical manner to generate a complete de-noised OCT-A volume. In still other embodiments, only one of the B-scan level and en-face level de-noising may be performed on an OCT-A volume to generate the de-noised OCT-A images and/or volume. Using the above process and example of four repeated scans per location, the result of de-noising is six de-noised OCT-A en-face images, B-scans, and/or volumes.

Referring back to FIG. 2, the above-described de-noised OCT-A volumes (and images therefrom) are than averaged, or otherwise statistically combined 204. In some embodiments, the processor 114 can determine an average (or like statistical combination) for OCT-A images or volumes with a short interscan time (SIT) (e.g., Δt) and a long interscan time (LIT) (e.g., greater than Δt) 204. In these cases, Δt represents a predetermined threshold separating a “short” interscan time from a “long” interscan time. For instance, in the above described example having six de-noised OCT-A en-face images, three de-noised OCT-A en-face images (from OCT B-scan pairs B1-B2, B2-B3, and B3-B4) have a SIT Δt, and three de-noised OCT-A en-face images (from OCT B-scan pairs B1-B3, B2-B4, and B1-B4) have a LIT greater than Δt. The processor 114 can thus determine an average of the three de-noised OCT-A en-face images of a SIT, generating a single en-face image that is representative of the SIT. The processor 114 can also determine an average of the three de-noised OCT-A en-face images of a LIT, generating a single en-face image that is representative of the LIT.

The relationship between the OCT-A images having a SIT and the OCT-A images having a LIT can be exploited to determine relative blood flow velocity. In one example embodiment, the processor 114 can determine relative blood flow velocity 205 based on a ratio between the SIT image and LIT image. For example a single en-face blood flow image may be generated by taking a pixel-wise ratio of the SIT-representative en-face image to the LIT-representative en-face image. This resulting en-face blood flow image can be analyzed and processed by the processor 114 to determine blood flow velocity and like related quantities. For example, the individual pixel values (the ratio values) of the en-face blood flow image may correspond to a relative blood flow velocity. These en-face blood flow images correspond to the depths at which the de-noised OCT-A en-face images are taken (and thus the SIT- and LIT-representative images represents). As the OCT-A en-face images may be de-noised at one more depths, the en-face blood flow images may also be at one or more depths. For example, en-face blood flow images may be generated at a superficial depth, a deeper depth (e.g., in the choroid), and at the choriocapillaris.

Because longer interscan times are more sensitive to slow flow speeds, and shorter interscan times are more sensitive to faster flow speeds, a smaller ratio value (smaller SIT numerator but greater LIT denominator) indicates a slower estimated blood flow, while a larger ratio value (larger SIT numerator but smaller LIT denominator) indicates faster blood flow. Of course, the inverse of the ratio (with an LIT numerator and SIT denominator) could also be utilized. In still other embodiments, the dynamic range of the estimated relative blood flow velocity determined from the en-face blood flow image can be improved by using a ratio to a power greater than 1. For example, the ratio may be taken to a power of 1.5. Increasing the dynamic range of the estimated relative blood flow velocity can allow for a greater range of values, and therefore more detailed estimates.

As noted above, each pixel of the en-face blood flow image may correspond to the relative blood flow velocity and be the ratio of the SIT-representative and LIT-representative images, or other statistical combination of the SIT- and LIT-representative images (e.g., the ratio taken to a power of 1.5). The processor 114 can further generate other types of images from en-face blood flow image, for example, B-scans, volumes, and the like.

These generated en-face blood flow images can be color-mapped, for example, depicting the relative blood flow velocity in different colors (e.g., blue for slower blood flow, red for faster blood flow). In other words, the relative blood flow velocity (e.g., the ratio value) is mapped to a hue of the pixel in the en-face blood flow image. Such images indicating blood flow velocity can be useful for alerting clinicians of the type of blood vessels, and identifying diseases such as ballooning and narrowing of vessels, and even leakage of vasculature.

In some embodiments, such as those in which the en-face blood flow image is in grayscale, the relative blood flow value (e.g., the ratio value) can be expressed as pixel intensity.

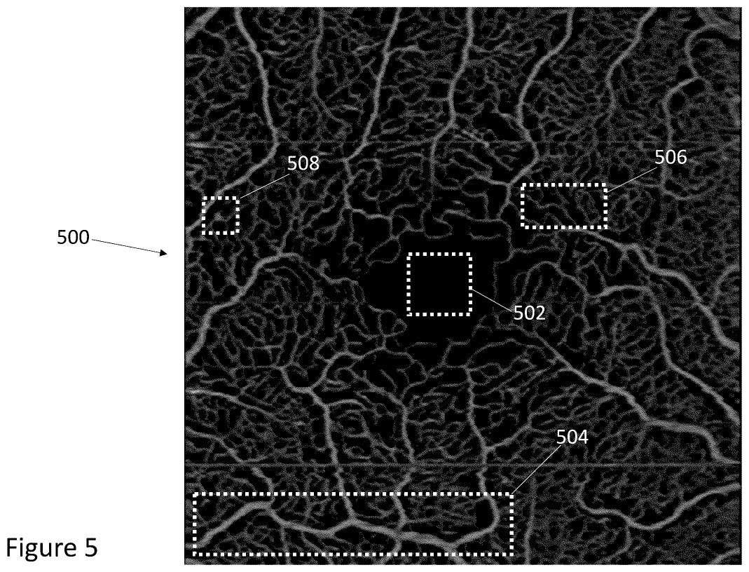

FIG. 5 depicts an example en-face blood flow image 500 in grayscale. The grayscale image can depict relative blood flow as light intensity, ranging from black at the weakest intensity (and least flow velocity) to white at the strongest (and greatest flow velocity). For example, location 502 in the en-face blood flow image 500 is the foveal avascular zone and is thus black, associated of the lack of relative blood flow. In contrast, location 504 contains vasculature depicted by higher intensity information associated with faster relative blood flow, and location 506 of the image 500 depicts vasculature with a medium intensity associated with slower relative blood flow. As mentioned above, these images are useful in identifying disease or problems with the vasculature. For example, location 508 of image 500 illustrates leakage of the vasculature. The color-mapped images can be displayed on a display or saved to computer-readable medium, such as random access memory (RAM) or a hard drive.

While various features are present above, it should be understood that the features may be used singly or in any combination thereof. Further, it should be understood that variations and modifications may occur to those skilled in the art to which the claimed examples pertain.

What is claimed is:

1. A method comprising:
generating at least three structural optical coherence tomography (OCT) images of a same location of an object;
generating at least two OCT-Angiography (OCT-A) images based on the structural OCT images, the at least two OCT-A images being based on different interscan times between the corresponding structural OCT images from which the OCT-A images were generated;
de-noising the at least two OCT-A images;
generating a short interscan time (SIT) representative image and a long interscan time (LIT) representative image based on the at least two OCT-A images;
estimating a relative blood flow velocity based on the SIT-representative image and the LIT-representative image.

2. The method of claim 1, wherein the at least two OCT-A images are cross-sectional B-scans.

3. The method of claim 2, further comprising:
generating the at least two OCT-A images for a plurality of locations of the object, thereby forming a plurality of OCT-A volumes; and
subsequent to de-nosing the at least two OCT-A images, de-noising an en-face image of each of the plurality of OCT-A volumes,
wherein the SIT-representative image and the LIT-representative image are based on the denoised en-face images.

4. The method of claim 1, further comprising generating a blood flow image based on the estimated relative blood flow velocity.

5. The method of claim 3, wherein the blood flow image is a color-mapped image in which pixel color corresponds to the estimated a relative blood flow velocity.

6. The method of claim 1, wherein the de-noising is performed by at least one trained machine learning system.

7. The method of claim 1,
wherein generating the SIT-representative image comprises statistically combining de-noised OCT-A images having an interscan time less than a predetermined threshold; and
wherein generating the LIT-representative image comprises statistically combining de-noised OCT-A images having an interscan time greater than the predetermined threshold.

8. The method of claim 1, wherein the estimated relative blood flow velocity at a given location is a ratio of the SIT-representative image at the given location to the LIT-representative image at the given location.

9. The method of claim 8, wherein estimating the relative blood flow velocity is a pixel-wise determination of the ratio of the SIT-representative image to the LIT-representative image.

10. The method of claim 8, wherein the ratio is raised to a power greater than or equal to 1.5.

11. The method of claim 1, wherein the object is a retina.

12. A method comprising:
generating a plurality of optical coherence tomography angiography (OCT-A) volumes, each of the plurality of OCT-A volumes being based on different interscan times between structural OCT images from which the OCT-A volumes were generated;
de-noising the plurality of OCT-A volumes by:
de-noising B-scan images from the plurality of OCT-A volumes; and
subsequent to de-noising the B-scan images, de-noising en-face images from the plurality of OCT-A volumes;
generating a short interscan time (SIT) representative image by statistically combining de-noised en-face images from OCT-A volumes having an interscan time less than a predetermined threshold; and
generating a long interscan time (LIT) representative image by statistically combining de-noised en-face images from OCT-A volumes having an interscan time greater than the predetermined threshold.

13. The method of claim 12, further comprising:
estimating a relative blood flow velocity based on the SIT-representative image and the LIT-representative image; and
generating a blood flow image based on the estimated relative blood flow velocity.

14. The method of claim 12, wherein the de-noising is performed by at least one trained machine learning system.

15. The method of claim 12, further comprising:
estimating a relative blood flow velocity as a pixel-wise determination of a ratio of the SIT-representative image at the given location to the LIT-representative image raised to a power greater than or equal to 1.5; and
generating a blood flow image based on the estimated relative blood flow velocity.

16. The method of claim 12, wherein the object is a retina.
